(19) United States
(12) Patent Application Publication
(10) Pub. No.: US 2002/0028222 A1
(43) Pub. Date: Mar. 7, 2002

(54) Use of fibers in a care composition or a make-up composition to make the skin matte

(75) Inventor: Isabelle Afriat, New York, NY (US)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: L'OREAL, Paris (FR)

(21) Appl. No.: 09/847,388

(22) Filed: May 3, 2001

(30) Foreign Application Priority Data
May 4, 2000 (FR) 0005712

(51) Int. Cl. A61K 7/00; A61K 35/78; A61K 31/717; A61K 31/7048

(52) U.S. Cl. 424/401; 514/28; 424/725; 514/171; 514/154; 514/440; 514/558; 514/57

(57) ABSTRACT

The present application relates to the use of fibers in a skincare composition or a make-up composition for the skin, to make the complexion matte, smooth and/or uniform, and/or to fade out skin relief defects. The fibers are in particular polyamide fibers having a length of from 1 μm to 10 mm and a shape factor of from 5 to 150. The composition used gives the skin a covering index of greater than 0.1 and preferably greater than 0.13. The invention also relates to a cosmetic treatment process for fading the complexion matte, smooth and/or uniform, and/or for fading out microreliefs, wrinkles, fine lines and pores in the skin, comprising the application to the skin of fibers in a cosmetic composition.

### FIELD OF THE INVENTION

The present application relates to the use of fibers in a skincare composition or a make-up composition for the skin, to make the complexion matte, smooth and/or uniform. The application also relates to a cosmetic treatment process aimed at making the complexion of the skin matte, smooth and/or uniform, and/or fading out the microreliefs, wrinkles and pores in the skin comprising the application to the skin of a cosmetic composition containing fibers.

### DISCUSSION OF THE BACKGROUND

Skincare compositions or make-up compositions having matte-effect properties are generally used to solve the sheen problems caused by excess sebum and to improve the long-term staying power of make-up, since make-up has a tendency to degrade visually in the course of the day. They give the skin a matte appearance, resulting from a power to scatter light at the surface of the skin. They may also be used to fade out skin defects such as microreliefs, wrinkles, fine lines and pores or color variations.

Conventional compositions, known as matte-effect compositions, generally contain very few fatty substances or are free of fatty substances. They generally consist of powders which absorb sebum and the excess oil of the composition which is not absorbed by the skin. Among the matte-effect powders of natural or synthetic origin which may be mentioned in particular are fillers such as talc, starch, mica, silica, Nylon powders, polyethylene powders, poly-beta-alanine and poly(methyl methacrylate). Fillers of this type have the drawback of not giving the skin a natural appearance, by giving a powdery or even plaster-like appearance and of accentuating defects in the skin. Furthermore, the compositions containing them generally have a drying effect in the long term and are difficult to spread. Their matte effect in not long-lasting.

Document EP-A-0 502 769 discloses matte-effect compositions which give a translucent layer and a natural appearance to made-up skin. They are dispersions of spherical particles in a fatty binder, in a highly specific filler/binder weight ratio. In order to have a matte effect, a high proportion of powders is required and, consequently, these compositions may cause drying-out. In addition, they have a tendency to form fluff during spreading and to give the skin a bleaching effect on account of the high concentration of powders.

Cosmetic compositions containing short polyamide fibers are known, for example from document JP 07-196 440, these fibers giving the said compositions a velvety feel and good cosmetic staying power. However, no document describes that the fibers can have a matte effect, and/or covering power, or that they can make it possible to fade out defects (wrinkles, fine lines or pores) in the skin (covering power).

### SUMMARY OF THE INVENTION

The Applicant has discovered, surprisingly, that fibers, and in particular polyamide fibers, can constitute a noteworthy matte-effect agent. Cosmetic compositions containing these fibers make it possible to make the complexion matte, smooth and/or uniform, while at the same time being soft to apply, easy to spread and non-sticky and not drying out the skin. They have good covering power on the skin and may be used in particular to fade out skin relief defects such as microreliefs, wrinkles and pores, while at the same time giving the skin a natural appearance.

Thus, one subject of the present invention is the use of fibers in a skincare composition, as an agent for making the complexion matte, smooth and/or uniform, and/or for fading out skin relief defects.

Another subject of the invention is the use of fibers in a skincare composition for making the complexion matt, smooth and/or uniform, and/or for fading out skin relief defects.

A subject of the invention is also the use of fibers in a cosmetic emulsion, as an agent for making the complexion matt, smooth and/or uniform, and/or for fading out skin relief defects, and the cosmetic use of fibers in a cosmetic emulsion, for making the complexion matt, smooth and/or uniform, and/or for fading out skin relief defects.

Another subject of the invention is the use of fibers in a skincare composition and/or a make-up composition for the skin, for fading out microreliefs, wrinkles fine lines and pores or color variations in the skin.

A subject of the invention is also a treatment process for making the complexion matte, smooth and/or uniform, and/or for fading out microreliefs, wrinkles, fine lines and pores in the skin, comprising the application to the skin of a cosmetic skincare composition containing fibers in a physiologically acceptable medium.

The invention also relates to a make-up process for microreliefs, wrinkles, fine lines and/or pores in the skin, which consists in applying to the skin a cosmetic composition containing fibers in a physiologically acceptable medium.

The composition used according to the invention contains fibers in an amount which is sufficient to produce a covering index of greater than 0.1 and preferably greeter than 0.13. The covering index is defined as being the ratio Yb/Yw (black clarity/white clarity) in the covering test described hereinbelow.

Another subject of the invention is the use of fibers in a skincare composition or a make-up composition for the skin, in an amount which is sufficient for the said composition to have a covering index of greater than 0.1.

### DETAILED DESCRIPTION OF THE INVENTION

The cosmetic composition containing the fibers contains a physiologically acceptable medium. The expression “physiologically acceptable medium” means herein a medium which is compatible with the skin, the lips, the scalp, the eyelashes, the eyes, the nails and/or the hair.

The fibers which may be used in the composition of the invention may be hydrophilic or hydrophobic fibers, of natural or synthetic, mineral or organic origin.

These fibers may be short or long, individual or organized, for example braided. They may have any shape or morphology and in particular may have a circular or polygonal (square, hexagonal or octagonal) cross section depending on the specific application envisaged. In particular, their ends are blunted and/or polished to prevent injury.

In particular, the fibers may have a length (L) ranging from 1 μm (0.001 mm) to 10 mm, preferably from 0.1 μm to 5 mm and better still from 0.1 mm to 1.5 mm. Their cross section may be within a circle of diameter (D) ranging from 1 nm (0.001 μm) to 100 μm, preferably ranging from 1 nm (0.001 μm) to 50 μm and better still from 5 μm to 40 μm.

Preferably, the fibers used according to the present invention have a shape factor, i.e. a ratio L/D (length/diameter) ranging from 3.5 to 2500, better still from 5 to 500 and even better still from 5 to 150.

The yarn court of the fibers is often given in denier or decitex. The denier is the weight in grams for 9 km of yarn. The fibers used according to the invention preferably have a yarn count ranging from 0.15 to 30 denier and better still from 0.18 to 18 denier.

The shape factor, the yarn count and the morphology of the fibers are the three factors that are important for defining a fiber.

The fibers may be those used in the manufacture of textiles and in particular silk, cotton, wool or flax fibers; cellulose fibers extracted in particular from wood, plants or algae; polyamide (NYLON®) fibers; modified cellulose (rayon or viscose or acetate, in particular rayon acetate) fibers; poly-p-phenyleneterephthalamide fibers, in particular KEVLAR® fibers; acrylic fibers, in particular polymethyl methacrylate or poly(2-hydroxyethyl methacrylate) fibers; polyolefin fibers and in particular polyethylene or polypropylene fibers; glass, silica or aramid fibers; carbon fibers, in particular in the form of graphite; TEFLON® fibers; insoluble collagen fibers; polyester, polyvinyl chloride, polyvinylidene chloride, polyvinyl alcohol, polyacrylonitrile, chitosan, polyurethane or polyethylene phthalate fibers; and fibers formed from a mixture of polymers such as those mentioned above, for instance polyamide/polyester fibers.

The resorbable synthetic fibers used in surgery may also be used, for instance the fibers prepared from glycolic acid and from caprolactone (MONOCRYL from the company Johnson & Johnson); resorbable synthetic fibers such as the copolymer of lactic acid and of glycolic acid (VICRYL from the company Johnson & Johnson); terephthalic polyester fibers (ETHIBOND from the company Johnson & Johnson) and stainless steel yarns (ACIER from the company Johnson & Johnson).

Moreover, the fibers may or may not be surface-treated and may be coated or uncoated. As coated fibers which mad be used in the invention, mention may be made of polyamide fibers coated with copper sulphide for an antistatic effect (for example R-STAT from the company Rhodia) or another polymer allowing a particular organization of the fibers (specific surface treatment) or a surface treatment which induces color/hologram effects (for example LUREX fiber from the company Sildorex).

A mixture of several kinds of fiber may be used.

According to their properties, the fibers used according to the present invention may be introduced into an aqueous medium, an oily medium or into a powder.

The fibers which may be used in the composition according to the invention are preferably chosen from polyamide fibers, poly-p-phenylene-terephthalamide fibers and cotton fibers, and mixtures thereof. Their length may range from 0.1 to 10 mm and preferably from 0.1 to 1 mm, their mean diameter may range from 5 to 50 μm and the shape factor preferably ranges from 5 to 150.

In particular, the polyamide fibers sold by Etablissements P. Bonte under the name POLYAMIDE 0.9 DTEX 0.3 mm, having a mean diameter of from 15 to 20 μm, a yarn count of about 0.9 dtex (0.81 denier) and a length ranging from 0.3 mm to 1.5 mm, may be used. Poly-p-phenyleneterephthalamide fibers with a mean diameter of 12 μm and length of about 1.5 mm may also be used, such as those sold under the name KEVLAR FLOC by the company Du Pont fibers. These polyamide fibers are preferably introduced into an oily medium or introduced via a dry route into a powder.

Cotton fibers with a mean diameter of 20 μm, a length of 0.3 mm and a shape factor of 15 may also be used, such as those sod by the company Filature de Lomme, by the company Textiles des dunes or by the company Velifil.

The fibers may be present in the composition according to the invention in an amount ranging from 0.1% to 30% by weight, preferably from 1% to 25% by weight and better stir from 5% to 25% by weight relative to the total weight of the composition. The amount of fibers to be used depends on the nature and cross sectional shape of the fibers used. Thus, for polyamide fibers, an amount of at least 5% fibers gives particularly satisfactory results in terms of the matte effect and the fading-out of skin defects. For microfibrils, an amount of 1% already gives good results.

The covering index makes it possible to characterize the covering power of a composition. To determine the covering index, a contrast card is prepared by spreading the composition, to a thickness of 150 μm, over a Byk-Gardener Penopac 1A contrast plate (140×254 mm) using a 50/100/150/300 micron 4-face multiple applicator. The film thus obtained is left to dry for about 24 hours.

A Minolta CR 200 colorimeter is calibrated using a special white calibration plate (Y=92; x=0.3138; y=0.3193. The ICI (International Commission on Illumination) diagram of the trichromatic coordinates makes it possible to characterize all the colors by means of flat coordinates, x and y, which define the chromaticity, i.e. the shade and saturation combination, and by means of a third coordinate Y defining the clarity. The apparatus is programmed to take 3 readings at each measurement, and automatically calculates the average, 2 measurements are taken on each part of the contrast card (black/white) and the values of Yb (Y black) and of Yw (Y white) are measured.

The covering index corresponds to the ratio Yb/Yw. The closer this ratio is to 1, the greater the covering power. For a covering index of 1, the covering is complete, i.e. a white effect is obtained on the skin, which is not desirable for a natural appearance of the matt effect. In order to have satisfactory covering and a good matte effect according to the present invention, the covering index should be greater than 0.1 and should preferably range from 0.1 to 0.5 and better still from 0.13 to 0.4.

The compositions according to the invention containing fibers may be in any presentation form conventionally used for topical application, and in particular in the form of anhydrous compositions, oily gels, aqueous or aqueous-alcoholic solutions, dispersions such as lotions or sera, aqueous gels, emulsions of liquid or semi-liquid consistency such as milks, obtained by dispersing a fatty phase in an aqueous phase (O/W) or conversely, (W/O), or suspensions or emulsions of soft, semi-solid or solid consistency such as creams or gels, or alternatively multiple emulsions (W/O/W or O/W/O emulsions), microemulsions, microcapsules, microparticles or vesicular dispersions of ionic and/or nonionic type. These compositions are prepared according to the usual methods.

According to one particular embodiment of the invention, the composition containing the fibers is a water-in-oil (W/O) or oil-in-water (O/W) emulsion. The proportion of the oily phase of the emulsion may range from 5% to 80°s by weight and preferably from 5% to 50% by weight relative to the total weight of the composition. The oils, emulsifiers and co-emulsifiers used in the composition in emulsion form are chosen from those used conventionally in cosmetics or dermatology. The emulsifier and the co-emulsifier are generally present in the composition in a proportion ranging from 0.3% to 30% by weight and preferably from 0.5% to 20% by weight relative to the total weight of the composition. The emulsion may also contain lipid vesicles.

As oils which may be used in the invention, mention may be made of mineral oils (liquid petroleum jelly), plant oils (liquid fraction or karite butter, sunflower oil), animal oils (perhydrosqualene), synthetic oils (hydrogenated polyisobutene), nonvolatile or volatile silicone oils (cyclomethicones such as cyclopentasiloxane) and fluoro oils (perfluoropolyethers). It is also possible to use, as fatty substances, fatty alcohols, fatty acids, waxes such as microcrystallize wax, jojoba wax, lanolin wax and beeswax. The oily phase of the emulsion may also contain gums such as silicone gum (dimethiconol), resins and in particular silicone resins such as trifluoromethyl (C₁₋₄) alkyldimethicone, and silicone elastomers such as the products sold under the name “KSG” by the company Shin-Etsu, under the name “Trefil” by the company Dow Corning or under the name “Gransil” by the company Grand Industries.

The emulsion generally contain at least one emulsifier chosen from amphoteric, anionic, cationic and nonionic emulsifiers, used alone or as a mixture. The emulsifiers are chosen appropriately according to the emulsion to be obtained (W/O or O/W emulsion).

For W/O emulsions, emulsifiers which may be mentioned, for example, include dimethicone copolyols such as the mixture of cyclomethicone and of dimethicone copolyol, sold under the name “DC 5225 C” by the company Dow Corning, and alkyldimethicone copolyols such as laurylmethicone copolyol sold under the name “Dow Corning 5200 Formulation Aid” by the company Dow Corning and cetyldimethicone copolyol sold under the name ABIL EM 90® by the company called Goldschmidt.

In a known manner, the cosmetic or dermatological composition of the invention may also contain adjuvants that are common in the cosmetics, pharmaceutical or dermatological fields, such as hydrophilic or lipophilic gelling agents, hydrophilic or lipophilic active agents, preserving agents, antioxidants, solvents, fragrances, fillers, screening agents, bactericides, odor absorbers, dyestuffs and salts. The amounts of (these various adjuvants are those that are conventionally used in the field under consideration, and, four example, from 0.01 to 10% relative to the total weight of the composition. Depending on their nature, these adjuvants may be introduced into the fatty phase, into the aqueous phase and/or into the lipid spherules. These adjuvants and their concentrations should be such that they do not modify the desired property for the composition of the invention.

As solvents which may be used in the invention, mention may be made of lower alcohols containing from 1 to 4 carbon atoms, in particular ethanol, isopropanol and propylene glycol.

Lipophilic gelling agents which may be mentioned include modified clays such as hectorite and its derivatives, for instance the products sold under the name BENTONE.

As fillers which may be used in the composition of the invention, mention may be made, for example, besides pigments, of silica powder; talc; polyamide particles and in particular those sold under the name ORGASOL by the company Atochem; polyethylene powders; microspheres based on acrylic copolymers, such as those made of ethylene glycol dimethacrylate/lauryl methacrylate, sold by the company Dow Corning under the name POLYTRAP; expanded powders such as hollow microspheres, and in particular the microspheres sold under the name EXPANCEL; by the company Kemanord Plast or under the name MICROPEARL F 80 ED by the company Matsumoto; powders of natural organic materials such as cornstarch, wheat starch or rice starch, which may or may not be crosslinked, such as starch powders crosslinked with octene succinate anhydride, sold under the name DRY-FLO by the company National Starch; silicone resin microbeads such as those sold under the name TOSPEARL by the company Toshiba Silicone; and mixtures thereof. These fillers may be present in amounts ranging from 0 to 40% by weight and preferably from 1% to 10% by weight relative to the total weight of the composition.

On account of the matte effect obtained with the composition used according to the invention, this composition may be used in particular for treating greasy skin, in particular to control the greasy appearance of the skim In this case, it may advantageously contain at least one active agent for treating greasy skin. This active agent may be chosen in particular from β-lactam derivatives, quinolone derivatives, ciprofloxacine, norfloxacine, tetracycline and its salts, erythromycin and its salts, amikacin and its salts, 2,4,4′-trichloro-2′-hydroxydiphenyl ether (or triclosan), 3,4,4′-trichlorocarbanilide (or triclocarban), phenoxypropanol, phenoxyisopropanol, doxycycline and its salts, capreomycin and its salts, chlorhexidine and its salts, chlortetracycline and its salts, oxytetracycline and its salts, clindamycin and its salts, ethanbutol and its salts, hexamidine isethionate, metronidazole and its salts, pentamidine and its salts, gentamicin and its salts, kanamycin and its salts, lineomycin and its salts, methacycline and its salts, methenamine and its salts, minocycline and its salts, neomycin and its salts, netilmicin and its salts, paromomycin and its salts, streptomycin and its salts, tobramycin and its salts, miconazole and its salts, amanfadine salts, para-chloro-metaxylenol, nystatin, tolnaftate, 3-hydroxybenzoic acid and its salts, 4-hydroxybenzoic acid and its salts, 2-hydroxybutanoic acid and its salts, 2-hydroxypentanoic acid and its salts, 2-hydroxyhexanoic acid and its salts, phytic acid and its salts, N-acetyl-L-cysteine acid and its salts, lipoic acid and its salts, azelaic acid and its salts, arachidonic acid and its salts, ibuprofen, naproxen, hydrocortisone, acetaminophen, resorcinol, octopirox, lidocaine hydrochloride, clotrimazole, 10-hydroxy-2-decanoic acid and its salts, zinc salts such as zinc gluconate, plant extracts from the ericacea family such as extracts of checkerberry wintergreen (or Gaultheria procumbens), sulphur-rich plant extracts such as extracts of white dead nettle (or Lamium album), and mixtures thereof.

This or these active agents may be present in an amount ranging, for example, from 0.0001% to 30% by weight, preferably from 0.001% to 20% by weight and better still from 0.01% to 15% by weight relative to the total weight of the composition.

Thus, a subject of the present invention is also a cosmetic composition comprising fibers and at least one active agent for treating greasy skin, chosen in particular from those indicated above, and the cosmetic use of such a composition for controlling the greasy appearance of the skin and/or for treating greasy skin.

### EXAMPLES

The examples which follow serve to illustrate the invention without, however, being limiting in nature. The names are, depending on the case, chemical names or CTFA (International Cosmetic Ingredient Dictionary and Handbook) names, and the amounts are in percentages by weight, except where otherwise mentioned.

Example 1: W/O emulsion

| | | |
|---|---|---|
| (1) | Cyclomethicone/disteardimonium hectorite/ | 3% |
| | alcohol (85/10/5 mixture sold under the name | |
| | BENTONE GEL VS-5V by Elementis Specialties) | |
| (2) | Sodium chloride | 0.7% |
| (3) | Trifluoromethyl (Cl-4)alkyldimethicone | 4% |
| | (FL-5 (X-22-819) sold by Shin Etsu) | |
| (4) | Nylon 12 (ORGASOL 2002 EEXTRA D NAT COS | 1.5% |
| | sold by Atochem) | |
| (5) | Polyamide fibres | 12% |
| | (Polyamide 0.9 dtex, 0.3 mm-from Société | |
| | Paul Bonte) | |
| (6) | Cyclomethicone/dimethicone copolyol | 10% |
| | (DC-5225 C sold by Dow Corning) | |
| (7) | Dimethicone/dimethiconol (DC 1503 sold by | 2.5% |
| | Dow Corning) | |
| (8) | Cyclopentasiloxane | 7% |
| (9) | Preserving agents | 1% |
| (10) | Glycerol | 5% |
| (11) | Ethanol | 5% |
| (12) | Water | qs |
| | | 100% |

Procedure:

The constituents (3), (7), (1), half of the cyclopentasiloxane (8) and the fibers (5) are mixed together with a spatula and the mixture is then treated twice in a three-roll mill. Separately, the DC-5225C is mixed with the other half of the cyclopentasiloxane, the resulting mixture is mixed with the mixture obtained above and this final mixture is homogenized to give the oily phase.

Separately, the aqueous phase is prepared by mixing together the constituents of this phase: water, glycerol, ethanol, salt, preserving agents. The emulsion is prepared by adding the aqueous phase to the oily phase portionwise with stirring.

The composition thus obtained has matte-effect properties which persist over time, and it gives the skin a natural appearance after application.

Test to determine the covering index:

The covering index of the composition of Example 1 and of similar compositions containing no fibers (placebo) or containing a reduced amount of fibers was determined:

| | | | | |
|---|---|---|---|---|
| | Ex. | Ex. | Ex. | Ex. |
| | with | with | with | with |
| | 6% | 8% | 10% | 12% |
| Placebo | fibers | fibers | fibers | fibers |

1) Compositions

| | | | | | |
|---|---|---|---|---|---|
| Compositions | | | | | |
| Trifluoromethyl | 4.5 | 4.3 | 4.2 | 4.1 | 4.0 |
| (Cl-4)alkyl- | | | | | |
| dimethicone | | | | | |
| DC 1503 | 2.8 | 2.7 | 2.6 | 2.6 | 2.5 |
| Bentone Gel VS-5V | 3.4 | 3.2 | 3.1 | 3.1 | 3.0 |
| Nylon 12 | 1.7 | 1.6 | 1.6 | 1.5 | 1.5 |
| Polyamide fibres | | 6.0 | 8.0 | 10.0 | 12.0 |
| Cyclopentasiloxane | 8.0 | 7.5 | 7.3 | 7.2 | 7.0 |
| DC-5225 C | 11.4 | 10.7 | 10.5 | 10.2 | 10.0 |
| Sodium chloride | 0.8 | 0.7 | 0.7 | 0.7 | 0.7 |
| Glycerol | 5.7 | 5.3 | 5.2 | 5.1 | 5.0 |
| Ethanol | 5.7 | 5.3 | 5.2 | 5.1 | 5.0 |
| Preserving Agents | 1.1 | 1.1 | 1.0 | 1.0 | 1.0 |
| Water | qs | qs | qs | qs | qs |
| | 100.0 | 100.0 | 100.0 | 100.0 | 100.0 |

2) Results

| | | | | | |
|---|---|---|---|---|---|
| Values obtained | | | | | |
| Average Y_{black} | 4.2600 | 10.9500 | 17.2800 | 24.0000 | 27.1600 |
| Average x_{black} | 0.3100 | 0.2940 | 0.2962 | 0.2987 | 0.3001 |
| Average y_{black} | 0.3170 | 0.3019 | 0.3040 | 0.3062 | 0.3073 |
| Average Y_{white} | 82.4700 | 82.4800 | 82.2400 | 82.4100 | 82.4700 |
| Average x_{white} | 0.3166 | 0.3168 | 0.3169 | 0.3168 | 0.3167 |
| Average y_{white} | 0.3222 | 0.3224 | 0.3225 | 0.3223 | 0.3222 |
| Y_{black}/Y_{white} | 0.0517 | 0.1328 | 0.2101 | 0.2912 | 0.3293 |

It emerges from the above table that the covering index is greater than 0.1 only for the compositions containing fibers and that it increases as the percentage of fibers contained in the formula increases.

Example 2: W/O emulsion

| | | |
|---|---|---|
| (1) | Microcrystalline wax | 1.41% |
| (2) | Hydrogenated polyisobutene | 5.44% |
| (3) | Propyl paraben (preserving agent) | 0.02% |
| (4) | Polyaminopropyl biguanide (preserving | 1% |
| | agent) | |
| (5) | Magnesium sulphate | 0.7% |
| (6) | Silica | 0.64% |
| (7) | Polyamide fibres 8.2 (Polyamide 0.9 dtex, 0.3 mm- | 8.2% |
| | from Societe Paul Bonte) | |
| (8) | Ethylene/acrylic acid copolymer | 0.7% |
| (9) | Acrylates copolymer | 0.05% |
| (10) | Cyclopentasiloxane | 20% |
| (11) | Cyclomethicone/dimethicone copolyol | 10% |
| | (DC-5225C sold by Dow Corning) | |
| (12) | Polymethylsesquioxane | 0.5% |
| (13) | Dimethicone/vinyl dimethicone | 3% |
| | crosspolymer/dimethicone | |
| | (KSG 16 sold by Shin-Etsu) | |
| (14) | Glycerol | 5% |
| (15) | Water | qs |
| | | 100% |

Procedure:

The constituents (1), (2), (3), (6), (8), (9), (12) and (13) are mixed together. The fibers (7) are added thereto with thorough mixing. The constituents (10) and (11) are then added and the oily phase thus obtained is homogenized. The emulsion is prepared in a Moritz blender by adding the aqueous phase obtained by mixing together the constituents (4), (5), (14) and (15) to the oily phase obtained above.

The composition obtained is capable of making the skin matt and of covering its imperfections (wrinkles and fine lines).

Test of matte-effect efficacy:

To demonstrate the matte effect of the composition according to the invention, the skin's sheen was evaluated over time after 15 minutes and 1 hour, observed on a sample of 17 individuals with greasy and shiny skin, onto whom were applied the matte-effect composition of Example 2 and, for comparison, an identical composition containing no fibers.

The matte-effect composition is applied to each individual, at a rate of 2 mg/cm², as a single application on one half of the forehead, the other half serving as the control area with a single application of the composition containing no fibers to one half of the forehead. Randomization is carried out to avoid area effects.

The climatic conditions are as follows:

Temperature: 22° C.

Relative humidity: 41%

At time T=Q (T₀), T=15 min and T=1 h, the sheen of the surface of the made-up skin (or of the untreated skin for the control area) is measured using a measuring device disclosed in the published application FR-2 650 890 and starting with the parallel-reflection and crossed-reflection parameters specific to this device, which make it possible to evaluate the sheen of the surface of the skin.

In a first stage, at time T, the variation in the average sheen measured on the treated area is calculated by the formula:

Δ₁=(S′_{T}−S′₀)/S₀

in which:

S₀ denotes the average sheen measured at T₀, and

S_{T} denotes the average sheen measured at T.

In a second stage, at time T, the variation in the average sheen measured on the control area (treated with the composition not containing fibre) is calculated by the formula:

Δ₂=(S_{T}−S′₀)/S′₀

in which:

S′₀ denotes the average sheen measured on the control area at T₀, and

S′_{T} denotes the average sheen measured on the control area at T.

The results of these tests are summarized in the following table:

| Time | 15 minutes | 1 hour |
|---|---|---|
| Variation of the average | −14% | −13% |
| sheen on the treated area | | |
| (Example 2) | | |
| Δ₁ | | |
| Variation of the average | −4% | −4% |
| sheen on the control area | | |
| (Composition without | | |
| fibers) | | |
| Δ₂ | | |

These results show that the fibers give the compositions containing them good matte-effect properties on the skin.

Example 3: Cast product

| | |
|---|---|
| Phase A1 | |
| Jojoba wax | 5.3% |
| Polyglyceryl-4 isostearate/cetyldimethicone | 2.2% |
| copolyol/hexyl laurate | |
| (ABIL WE 09 sold by Goldschmidt) | |
| Cetearyl octanoate/isopropyl myristate | 3.1% |
| Polyethylene | 0.7% |
| Phase A2 | |
| Cyclohexasiloxane | 3.1% |
| Phase B | |
| Sodium chloride | 0.5% |
| Gycerol | 2% |
| Preserving agents | 1% |
| Water | qs |
| | 100% |
| Phase C | |
| Cyclomethicone/disteardimonium hectorite/ | 1.1% |
| alcohol (85/10/5 mixture sold under the name | |
| BENTONE GEL VS-5V by Elementis Specialties) | |
| Trifluoromethyl (Cl-9)alkyldimethicone | 2.1% |
| (FL-5 (X-22-819) sold by Shin Etsu) | |
| Nylon 12 (ORGASOL 2002 EXTRA D NAT COS sold | 1% |
| by Atochem) | |
| Polyamide fibres (Polyamide 0.9 dtex, | 8% |
| 0.3 mm-from Societe Paul Bonte) | |
| Dimethicone/dimethicanol (DC 1503 sold by | 1.3% |
| Dow Corning) | |

Procedure:

Phase C is prepared by mixing together all the constituents and treating the mixture three times in a three-roll mill. The aqueous phase (B) (sodium chloride+glycerol+water+preserving agents) is placed on a water bath at 90° C. Phase A1 is melted by stirring the mixture of constituents with a spatula at 80-85° C. (until completely liquefied). Phase A2 is then added to phase A1, after which the mixture is placed on a water bath at 80-85° C. with continued stirring.

The emulsion is prepared by adding phase B to the mixture of A1 and A2, at 85° C. with stirring. Phase C is then added thereto at about 70-75° C. by mixing together for about 2 minutes, with continued stirring.

Once the emulsion is prepared, it is cast in dishes while hot and left to cool.

A care composition in the form of a compact cast product which is applied with a sponge is obtained.

The composition was tested on a panel of 5 individuals. It was found to be of pleasant texture, easy to spread, comfortable to wear and to leave the skin soft and non-sticky after application. In addition, a lightening, smoothing and matte effect on the treated skin was noted.

Example 4: Powder

| | |
|---|---|
| Talc | 75.3% |
| Polyamide fibres-polyamide 0.9 d.tex, | 7.5% |
| 0.3 mm (Societe Paul Bonte) | |
| Nylon powder | 10% |
| Pigments | 3% |
| Silicone binder | 4% |
| Preserving agent | 0.2% |

Procedure:

All of the compounds, except the fibers, are mixed together in a Baker Perkins machine. The silicone binder is a mixture of polymethyl/cetyldimethylsiloxane (ABIL WAX 9801 from the company Goldschmidt), of polydimethylsiloxane/trimethylsiloxysilicate resin (Dow Corning Fluid 593) and of polydimethylsiloxane 10 cSt (Dow Corning Fluid 200). The fibers are added with stirring at the end of preparation in a Hosokawa Alpine mill.

The composition obtained presents, when used as make-up on a panel of 16 women:

a matte/satin glint,

good uniformity,

a corrective effect

Example 5: Lipstick

| | |
|---|---|
| Lanolin wax | 5% |
| Microcrystalline wax | 11% |
| Modified beeswax | 4.5% |
| Arara oil | 21% |
| Sesame oil | 22% |
| Modified clay | 0.6% |
| Acetylated lanolin | 6% |
| Cotton fibre (0.3 mm long) | 5% |
| Pigments | 9% |
| Antioxidant | 0.1% |
| Lanolin | qs |
| | 100% |

A glossy, uniform stick of lipstick is obtained. When applied on a panel of 24 individuals, this film of lipstick leads to a significant reduction in gloss compared with the film obtained with a lipstick containing no fibers.

The disclosure of the priority document, French Application No. 0005712 filed May 4, 2000, is incorporated by reference in its entirety herein.

While the present invention has been described with respect to specific embodiments, it is not confined to the specific details set forth, but includes various changes and modifications that may suggest themselves to those skilled in the art, all falling within the scope of the invention as defined by the following claims.

What is claimed is:

1. A cosmetic, skincare or make-up composition containing fibers and at least one active agent for treating greasy skin.

2. The composition according to claim 1, wherein the at least one active agent is selected from the group consisting of β-lactam derivatives; quinolone derivatives; ciprofloxacine; norfloxacine; tetracycline and its salts; erythromycin and its salts; amikacin and its salts; 2,4,4′-trichloro-2′-hydroxydiphenyl ether; 3,4,4′-trichlorocarbanilide; phenoxypropanol; phenoxyisopropanol; doxycycline and its salts; capreomycin and its salts; chlorhexidine and its salts; chlortetracycline and its salts; oxytetracycline and its salts; clindamycin and its salts; ethambutol and its salts; hexamidine isethionate; metronidazole and its salts; pentamidine and its salts; gentamicin and its salts; kanamycin and its salts; lineomycin and its salts; methacycline and its salts; methenamine and its salts; minocycline and its salts; neomycin and its salts; netilmicin and its salts; paromomycin and its salts; streptomycin and its salts; tobramycin and its salts; miconazole and its salts; amanfadine salts; para-chloro-meta-xylenol; nystatin; tolnaftate; 3-hydroxybenzoic acid and its salts; 4-hydroxybenzoic acid and its salts; 2-hydroxybutanoic acid and its salts; 2-hydroxypentanoic acid and its salts; 2-hydroxyhexanoic acid and its salts; phytic acid and its salts; N-acetyl-L-cysteine acid and its salts; lipoic acid and its salts; azelaic acid and its salts; arachidonic acid and its salts; ibuprofen; naproxen; hydrocortisone; acetaminophen; resorcinol; octopirox; lidocaine hydrochloride; clotrimazole; 1 0-hydroxy-2-decanoic acid and its salts; zinc salts; plant extracts from the ericacea family; sulphur-rich plant extracts; and mixtures thereof.

3. The composition according to claim 1, wherein the amount of the at least one active agent ranges from 0.0001% to 30% by weight relative to the total weight of the composition.

4. The composition according to claim 1, wherein each of the fibers has a length in a range from 1 μm to 10 mm.

5. The composition according to claim 1, wherein each of the fibers has a cross section that fits within a circle having a diameter in a range from 1 nm to 100 μm.

6. The composition according to claim 1, wherein each of the fibers has a length L; a cross section that fits within a circle of diameter D; and a shape factor, given by L/D, that ranges from 5 to 150.

7. The composition according to claim 1, wherein the fibers are selected from the group consisting of silk, cotton, wool or flax fibers; cellulose fibers extracted from wood, plants or algae; polyamide fibers; modified cellulose fibers; poly-phenyleneterephthalamide fibers; acrylic, polyolefin, glass, silica, aramid, carbon or TEFLON® fibers; insoluble collagen fibers; polyester, polyvinyl chloride, polyvinylidene chloride, polyvinyl alcohol, polyacrylonitrile, chitosan, polyurethane or polyethylene phthalate fibers; fibers formed from a mixture of polymers; resorbable synthetic fibers; and mixtures thereof.

8. The composition according to claim 1, wherein the fibers are selected from the group consisting of polyamide fibers, poly-p-phenyleneterephthalamide fibers, cotton fibers, and mixtures thereof.

9. The composition according to claim 1, wherein the fibers are present in an amount ranging from 0.1% to 30% by weight relative to the total weight of the composition.

10. The composition according to claim 1, wherein the fibers have a yarn count ranging from 0.15 to 30 denier.

11. The composition according to claim 1, wherein the fibers are coated fibers.

12. The composition according to claim 1, wherein the composition is an emulsion.

13. The composition according to claim 1, wherein the composition has a covering index of greater than 0.1.

14. The composition according to claim 1, wherein the composition is a physiologically acceptable medium.

15. The composition according to claim 1, wherein the composition is in the form of a cast product.

16. A method of making a cosmetic, skincare or make-up composition, the method comprising
mixing fibers and at least one active agent for treating greasy skin; and
forming the composition of claim 1.

17. A method of using a cosmetic, skincare or make-up composition, the method comprising applying the composition of claim 1 to skin.

18. A method of using a cosmetic, skincare or make-up composition, the method comprising
applying the composition of claim 1 to skin; and
making the complexion of the skin at least one of matte, smooth and uniform, or fading out skin relief defects in the skin.

19. A method of using a cosmetic, skincare or make-up composition, the method comprising
applying the composition of claim 10 to skin; and
making the complexion of the skin at least one of matte, smooth and uniform, or fading out skin relief defects in the skin.

20. A method of using a cosmetic, skincare or make-up composition, the method comprising
applying the composition of claim 14 to skin; and
making the complexion of the skin at least one of matte, smooth and uniform, or fading out skin relief defects in the skin.

21. A method for changing the appearance of skin, the method comprising applying to the skin a composition containing fibers in a physiologically acceptable medium.

22. The method according to claim 21, wherein the composition gives the skin at least one of a matte appearance, a smooth appearance and a uniform appearance.

23. The method according to claim 21, wherein the composition fades out at least one of microreliefs, wrinkles, fine lines, pores and color variations in the skin.

24. The method according to claim 21, wherein each of the fibers has a length in a range from 1 μm to 10 mm.

25. The method according to claim 21, wherein each of the fibers has a cross section that fits within a circle having a diameter in a range from 1 nm to 100 μm.

26. The method according to claim 21, wherein each of the fibers has a length L; a cross section that fits within a circle of diameter D; and a shape factor, given by L/D, that ranges from 5 to 150.

27. The method according to claim 21, wherein the fibers are selected from the group consisting of silk, cotton, wool or flax fibers; cellulose fibers extracted from wood, plants or algae; polyamide fibers; modified cellulose fibers; poly-phenyleneterephthalamide fibers; acrylic, polyolefin, glass, silica, aramid, carbon or TEFLON® fibers; insoluble collagen fibers; polyester, polyvinyl chloride, polyvinylidene chloride, polyvinyl alcohol, polyacrylonitrile, chitosan, polyurethane or polyethylene phthalate fibers; fibers formed from a mixture of polymers; resorbable synthetic fibers; and mixtures thereof.

28. The method according to claim 21, wherein the fibers are selected from the group consisting of polyamide fibers, poly-p-phenyleneterephthalamide fibers, cotton fibers, and mixtures thereof.

29. The method according to claim 21, wherein the fibers are present in the composition in an amount ranging from 0.1% to 30% by weight relative to the total weight of the composition.

30. The method according to claim 21, wherein the fibers have a yarn count ranging from 0.15 to 30 denier.

31. The method according to claim 21, wherein the fibers are coated fibers.

32. The method according to claim 21, wherein the composition is an emulsion.

33. The method according to claim 21, wherein the composition has a covering index of greater than 0.1.

34. The method according to claim 21, wherein the composition is a skincare composition.
